(19) United States
(12) Patent Application Publication
(10) Pub. No.: US 2010/0297040 A1
(43) Pub. Date: Nov. 25, 2010

(54) Cellular age advantage; nutrient reservoir cosmetic composition

(76) Inventor: Candace Rose Keefe, Laguna Beach, CA (US)

Correspondence Address: Apriori Beauty, 26651 Cabot Road, Suite A, Laguna Hills, CA, 92653, US

(21) Appl. No.: 12/662,168

(22) Filed: Apr. 2, 2010

Related U.S. Application Data
(60) Provisional application No. 61/216,697, filed on May 20, 2009.

(51) Int. Cl. A61K 8/92; A61K 8/19; A61K 8/34; A61K 8/60; A61K 8/73; A61Q 17/04; A61K 8/97; A61K 8/37; A61K 8/365; A61K 8/63; A61K 8/36; A61K 8/44; A61K 8/67; A61K 8/35; A61K 8/49; A61Q 19/08

(52) U.S. Cl. 424/59; 424/600; 514/738; 514/53; 514/23; 514/54; 514/62; 514/725; 514/546; 514/552; 514/557; 514/171; 514/558; 514/560; 514/556; 514/458; 424/94.1; 514/689; 514/733; 514/263.34; 514/47; 514/456; 424/766; 424/769; 424/757

(57) ABSTRACT

In recent years the demand for cosmetic compositions and cosmetic methods for improving the appearance and condition of skin has grown enormously; consumers are seeking topical skin care products that help treat or delay the signs of natural (chronoaging) and environmental aging. The present invention relates to topical compositions for application to human skin and to their use in improving the condition and appearance of skin. It is based on the premise that effective treatment of normal skin conditions due to natural or environmental aging, such as wrinkles, lines, sagging, hyperpigmentation and age spots, and/or of sensitive, dry, rough, flaky, red, itchy, irritated skin may be obtained through the application of cosmetic compositions to the skin which comprise a combination of collagen stimulators, phytoestrogens, plant derived anti-inflammatory agents, fatty acid, antioxidants.

### CROSS-REFERENCE TO RELATED APPLICATIONS

Not applicable

### STATEMENT REGARDING FEDERALLY SPONSORED RESEARCH

Not applicable

### REFERENCE TO SEQUENCE LISTING, A TABLE, OR A COMPUTER PROGRAM LISTING COMPACT DISC APPENDIX

Not applicable

### FIELD OF THE INVENTION

This invention relates to topical compositions for application to human skin and to their use in improving the condition and appearance of skin.

### BACKGROUND OF THE INVENTION

Skin is subject to deterioration through dermatological disorders, environmental abuse (wind, air conditioning, central heating) or through the normal aging process (chronoaging), which may be accelerated by exposure of skin to sun (photoaging). In recent years the demand for cosmetic compositions and cosmetic methods for improving the appearance and condition of skin has grown enormously.

Consumers are increasingly seeking “anti-aging” cosmetic products which treat or delay the visible signs of chronoaging and photoaging skin such as wrinkles, lines, sagging, hyperpigmentation and age spots.

Consumers also frequently seek other benefits from cosmetic products in addition to anti-aging. The concept of “sensitive skin” has also raised the consumer demand for cosmetic products which improve the appearance and condition of sensitive, dry, rough and/or flaky skin and to soothe red, irritated and/or itchy skin. Consumers also desire cosmetic products which treat spots, pimples, blemishes etc.

There continues to be a need, however, for alternative effective cosmetic compositions for topical application to skin for treating/delaying the visible signs of aging and photodamaged skin such as wrinkles, lines, sagging, hyperpigmentation and age spots. Compositions and cosmetic methods which provide other skin care benefits in addition to anti-aging such as for improving the appearance and condition of dry, rough and flaky skin and to soothe irritated and itchy skin are particularly desirable.

We have now surprisingly found that effective treatment and prevention of normal skin conditions due to natural or environmental aging, such as wrinkles, lines, sagging, hyperpigmentation and age spots, and/or of sensitive, dry, rough, flaky, red, itchy, irritated skin may be obtained through the application of cosmetic compositions to the skin which comprises collagen stimulators, phytoestrogens, plant derived anti-inflammatory agents, fatty acid, antioxidants.

### SUMMARY OF THE INVENTION

The present invention is based on the premise that effective treatment and prevention of normal skin conditions due to natural or environmental aging, such as wrinkles, lines, sagging, hyperpigmentation and age spots, and/or of sensitive, dry, rough, flaky, red, itchy, irritated skin may be obtained through the application of cosmetic compositions to the skin which comprise a combination of collagen stimulators, phytoestrogens, plant derived anti-inflammatory agents, fatty acid, antioxidants.

### BRIEF DESCRIPTION OF THE SEVERAL VIEWS OF THE DRAWING

Not applicable

### DETAILED DESCRIPTION OF THE INVENTION

According to a first aspect of the present invention there is provided a topical composition comprising:

(a) three or more of following of collagen stimulators, phytoestrogens, plant derived anti-inflammatory agents, fatty acid, antioxidants. Preferably, the combination are derived from the mixture of following: retinol, retinyl acetate, retinyl palmitate, (or other retinyl esters), sucrose, sodium hyaluronare, mannose, trihalose, fructose, N-Acetyl glucosamine, Adenosine Triphospahte (ATP), caffeine, cholesterol, phytosterol, Phytosteroyl canola glyceride, jajoba oil, palmitic acid, stearic acid, oleic acid, carnithine, CoQ-10, resveratrol, acetyl resvaretrol, isoflavones (from soy, black cohosh, red cover), grape extract, olive extract, glycyrrhetinic acid, licorice extract.
(b) a dermatologically acceptable vehicle.

Such compositions are particularly useful for topical application to human skin for cosmetically treating/preventing skin conditions selected from the group consisting of wrinkling, sagging, photodamaged skin, sensitive skin, dry skin, rough skin, flaky skin, red skin, irritated skin, itchy skin and age spots. The compositions are also useful for application to the skin as a cosmetic treatment for spots, pimples and blemishes or as a cosmetic skin care product which promotes dermal repair and/or boosts collagen deposition in skin.

According to a second aspect the present invention provides a cosmetic method of treating/preventing skin conditions selected from the group consisting of wrinkling, sagging, photodamaged skin, sensitive skin, dry skin, rough skin, flaky skin, red skin, irritated skin, itchy skin and age spots, the method comprising applying to the skin a topical composition as described above.

In a further aspect, the invention also provides the use of retinyl esters, hyaluronic acid derivatives, and fatty acids, and/or derivatives thereof comprising retinyl esters, hyaluronic acid derivatives, and fatty acids moieties, in a topical composition for treating/preventing skin conditions selected from the group consisting of wrinkling, sagging, photodamaged skin, dry skin, rough skin, flaky skin, sensitive skin, irritated skin, itchy skin and age spots, wherein at least 50% by weight of the retinyl esters, hyaluronic acid derivatives, and fatty acids.

The inventive compositions and methods thus provide anti-aging benefits which result in the promotion of smooth and supple skin with improved elasticity and a reduced or delayed appearance of wrinkles and aged skin with improved skin color. A general improvement in the appearance, texture and condition, in particular with respect to the radiance, clarity, and general youthful appearance of skin is achieved. The inventive compositions and methods are also beneficial for soothing and calming sensitive, dry, rough, irritated, red, flaky and itchy skin. Thus the inventive methods and compositions advantageously provide a wide range of skin care benefits.

The term “treating” as used herein includes within its scope reducing, delaying and/or preventing the above mentioned skin conditions such as wrinkled, aged, photodamaged, dry and/or irritated skin and generally enhancing the quality of skin and improving its appearance and texture by preventing or reducing wrinkling and increasing flexibility, firmness, smoothness, suppleness and elasticity of the skin. The cosmetic compositions, methods and the uses of the collagen stimulators, phytoestrogens, plant derived anti-inflammatory agents, fatty acid, and antioxidants according to the invention may be useful for treating skin which is already in a wrinkled, aged, photodamaged, dry and irritated condition or for treating youthful skin to prevent or reduce those aforementioned deteriorative changes due to the normal aging/photo aging process.

What is claimed is:

1. A skin care composition comprising collagen stimulators, hydrating molecules, lipid barrier former, antioxidants, cellular energy modifier, mild estrogen mimicker and/or anti-inflammatory agents.

2. The skin care composition of claim J, wherein said collagen stimulators are retinol, retinyl acetate, retinyl palmitate, or hydroxyl acids (sal acid, AHA, etc)

3. The skin care composition of claim 1, wherein said collagen stimulator are, by weight, from about 0.01% to about 2.0% of said composition, preferably at 0.1 to 0.75%.

4. The skin care composition of claim 1, wherein said hydrating molecules are water, glycerin, butylene glycol, sucrose, sodium hyaluronare, mannose, trihalose, fructose, or N-Acetyl glucosamine.

5. The skin care composition of claim 1, wherein said hydrating molecules are, by weight, from about 0.01% to about 2.0% of said composition, preferably at 0.1 to 0.1%.

6. The skin care composition of claim 1, wherein said lipid barrier former are cholesterol, Phytosteroyl canola glyceride, jajoba oil, palmitic acid, stearic acid, oleic acid, and/or triolein

7. The lipid barrier former can also be actives that stimulate fatty synthesis metabolically such as carnithine.

8. The skin care composition of claim 1, wherein said barrier formers are, by weight, from about 0.01% to about 2.0% of said composition, preferably at 0.1 to 0.75%.

9. The skin care composition of claim 1, wherein said antioxidants are vitamin E, CoQ-10, resveratrol, acetyl resvaretrol or ubiquinones.

10. The skin care composition of claim 1, wherein said antioxidants are, by weight, from about 0.01% to about 2.0% of said composition, preferably at 0.1 to 0.75%.

11. The skin care composition of claim 1, wherein said cellular energy modifiers are Adenosine Triphospahte (ATP), caffeine, or other xanthene analogs.

12. The skin care composition of claim 1, wherein said cellular energy modifiers are, by weight, from about 0.01% to about 2.0% of said composition, preferably at 0.1 to 0.75%.

13. The skin care composition of claim 1, wherein said mild estrogenic modifiers are isoflavones (from soy, black cohosh, red cover), grape extract, or olive extract,

14. The skin care composition of claim 1, wherein said mild estrogenic modifiers are, by weight, from about 0.01% to about 2.0% of said composition, preferably at 0.1 to 0.5%.

15. The skin care composition of claim 1, wherein said anti-inflammatory agent are glycyrrhetinic acid, licorice extract, or phytosterols (such as betalunic acid)

16. The skin care composition of claim 1, wherein said collagen stimulator are, by weight, from about 0.01% to about 2.0% of said composition, preferably at 0.1 to 0.5%.

17. A skin care composition that consists of day cream or lotion containing 70% of composition 1 with sunscreen

18. A skin care composition that consists of night cream or lotion containing 70% of composition 1

19. A skin care composition that consists of an eye serum containing 70% of composition 1.

20. A skin care composition that consists of a spot treatment containing 70% of composition 1.
